(19) United States
(12) Patent Application Publication
(10) Pub. No.: US 2006/0020035 A1
(43) Pub. Date: Jan. 26, 2006

(54) Bone marrow protection with N-acetyl-L-cysteine

(75) Inventors: Edward A. Neuwelt, Portland, OR (US); Leslie L. Muldoon, Tigard, OR (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Oregon Health & Science University, Portland, OR (US)

(21) Appl. No.: 11/078,013

(22) Filed: Mar. 11, 2005

Related U.S. Application Data
(60) Provisional application No. 60/552,425, filed on Mar. 11, 2004.

(51) Int. Cl. A61K 31/198

(52) U.S. Cl. 514/562

(57) ABSTRACT

The present invention provides methods for preventing or ameliorating chemotherapeutic agent-induced bone marrow toxicity. These methods comprise administering an effective amount of N-acetyl-L-cysteine (L-NAC), alone or in combination with other agents, to a subject in need thereof. The present invention also provides compositions for preventing or ameliorating chemotherapeutic agent-induced bone marrow toxicity that comprises L-NAC.

### CROSS-REFERENCE TO RELATED APPLICATION

This application claims the benefit under 35 U.S.C. § 119(e) of U.S. Provisional Patent Application No. 60/552,425, filed Mar. 11, 2004, which this provisional application is incorporated herein by reference in its entirety.

### STATEMENT OF GOVERNMENT INTEREST

This invention was made with government support under Contract No. NS34608 awarded by National Institutes of Health. The government has certain rights in this invention.

### BACKGROUND OF THE INVENTION

1. Field of the Invention

The present invention is directed to a method for preventing or ameliorating chemotherapeutic agent-induced bone marrow toxicity. In particular, the invention is directed to administering N-acetyl-L-cysteine (L-NAC), alone or in combination with other agents, to prevent or ameliorate such a side effect.

2. Description of the Related Art

Chemotherapy causes numerous toxic side effects, including bone marrow toxicity, mucositis, liver and kidney toxicity, and ototoxicity. Bone marrow toxicity can force a dose reduction, reducing chemotherapy efficacy and can also cause major morbidity, even death in patients.

Current treatments to reduce bone marrow side effects include recombinant growth factors that are lineage-specific. Such growth factors include EPO (erythropoietin) for red cells and G-CSF (granulocyte colony stimulating factor) or GM-CSF (granulocyte macrophage colony stimulating factor) for various lineages of white cells. However, such growth factors act to stimulate lineage specific precursor cells to divide and mature down lineage-specific paths. Thus, the use of growth factors results in a more rapid recovery from bone marrow toxicity but does not generally reduce the nadir of toxicity. Such growth factors have been able to allow a patient to tolerate a greater number of cytotoxic treatments, but generally not higher doses of the cytotoxic agent administered.

The present invention meets the need for developing more effective bone marrow protection against chemotherapy and further provides other related advantages.

### BRIEF SUMMARY OF THE INVENTION

The present invention provides methods for preventing or ameliorating chemotherapeutic agent-induced toxicity, such as bone marrow toxicity. Such methods comprise administering to a patient in need thereof an effective amount of L-NAC. It was discovered by the present inventors that only the L-form, not the D-form of N-acetylcysteine, is effective in ameliorating chemotherapeutic agent-induced bone marrow toxicity. In certain embodiments, the methods of the present invention do not adversely affect the efficacy of the chemotherapeutic agents.

According to the present invention, L-NAC may be administered intravenously, intra-arterially, intra-peritoneally, orally, intradermally, subcutaneously, or transdermally. In certain embodiments, the L-NAC is administered intra-arterially, such as via the descending aorta.

In certain embodiments, L-NAC is administered prior to the administration of the chemotherapeutic agent or at least one of the chemotherapeutic agents. In other embodiments, L-NAC is administered concurrently with the administration of the chemotherapeutic agent or at least one of the chemotherapeutic agents. In certain embodiments, L-NAC is administered following the administration of the chemotherapeutic agent or at least one of the chemotherapeutic agents. For instance, L-NAC may be administered at least about 15 minutes, 30 minutes, 45 minutes, 1 hour, 1.5 hours, 2 hours, 3 hours or 4 hours prior to the administration of the chemotherapeutic agent(s).

In certain embodiments, L-NAC may be administered in conjunction with one or more other thiol-based compounds. In certain embodiments, the thiol-based compounds have a free radical scavenging activity. The thiol-based compounds may be selected from a group consisting of sodium thiosulfate, glutathione ethyl ester, D-methionine, S-adenosyl-methionine, cysteine, N,N′-diacetyl-cysterine, cystathione, glutathione, glutathione ethyl ester, glutathione diethyl ester, S-(1,2-dicarboxyethyl) glutathione triester, cysteamine, cysteine isopropylester, thiol amifostine and combinations thereof. In certain embodiments, the thiol-based compound or composition is sodium thiosulfate.

In certain embodiments, the methods of the present invention further comprise administering an effective amount of sodium thiosulfate (STS). STS may be administered intravenously or intra-arterially. It may be administered prior to, concurrent with, or subsequent to, the administration of chemotherapeutic agent(s). The dosage of administrating STS may be at least about 1, 2, 3, 4, 5, 6, 7, 8, 9, 10, 11, 12, 13, 14, 15, 16, 17, 18, 19, or 20 g/m² in humans. In addition, multiple doses (e.g., 1, 2, 3, 4, 5, 6, 8, or 10) may be used.

The chemotherapeutic agent may be any compound that is administered to a mammalian subject to destroy, or otherwise adversely affect, cancer cells. Such agents may be platinum derivatives, taxanes, steroid derivatives, anti-metabolites, plant alkaloids, antibiotics, arsenic derivatives, intercalating agents, alkylating agents, enzymes, biological response modifiers and combinations thereof. In certain embodiments, the chemotherapeutic agents are alkylating agents, such as platinum-containing alkylating agents. Exemplary platinum-containing alkylating agents include cisplatin, carboplatin, oxyplatin, or combinations thereof. In certain embodiments, the chemotherapeutic agents comprise melphalan, carboplatin and etoposide phosphate.

A patient in need of prevention or amelioration of chemotherapeutic agent-induced bone marrow toxicity may be a human, a non-human primate, or another mammal that will undergo (or is undergoing) chemotherapy and is at high risk for (or is suffering from) chemotherapeutic agent-induced bone marrow toxicity. In certain embodiments, the patient may suffer from a tumor in the head or neck (e.g., brain tumor or cancer). In other embodiments, the patient may suffer from a tumor or cancer located other than head or neck. In certain embodiments, the patient receives a blood brain barrier disruption procedure. In other embodiments, the patient does not receive a blood brain barrier disruption procedure.

The dosage of L-NAC useful in preventing or ameliorating bone marrow toxicity may be about 200, 300, 400, 500, 600, 700, 800, 900, 1000, 1100, 1200, 1300, or 1400 mg/kg in humans. In addition, multiple doses (e.g., 1, 2, 3, 4, 5, 16, 8, 9, 10, etc.) may be used.

In one embodiments, the methods of the present invention comprises (i) administering to a patient in need thereof about 1,000 mg/kg to about 1,400 mg/kg of L-NAC about 30 minutes or about 60 minutes prior to the administration of chemotherapeutic agent(s) and (ii) administering about 8 to about 20 g/m² of STS about 4 hours and/or about 8 hours subsequent to the administration of the chemotherapeutic agent(s).

In another aspect, the present invention also provides a composition that comprises L-NAC and a pharmaceutically acceptable carrier, adapted for preventing or ameliorating bone marrow toxicity including thrombocytopenia, wherein the composition does not contain the same amount of D-NAC.

### BRIEF DESCRIPTION OF THE SEVERAL VIEWS OF THE DRAWINGS

FIG. 1 shows N-Acetylcysteine clearance from rat blood. Normal Long Evans rats received N-acetylcysteine as follows: 1200 mg/kg aortic infusion (n=3) (A), 1000 mg/kg aortic infusion (n=3) (B), 400 mg/kg intravenously (n=4) (C), and 140 mg/kg aortic infusion (n=2) (D). Blood samples were collected at the indicated times after the end of the infusion, and N-acetylcysteine concentrations (millimolar) were evaluated using a calorimetric kit.

FIGS. 2A-2C show chemoprotection for hematological toxicity. Nude rats with intracerebral tumors were treated with chemotherapy alone or in combination with chemoprotection consisting of N-acetylcysteine (1000 mg/kg, aortic infusion) 60 min before chemotherapy and/or sodium thiosulfate (8 g/m², intravenous administration) 4 and 8 h after tri-drug chemotherapy. Six days after treatment, blood counts were determined for total white cells (FIG. 2A), granulocytes (FIG. 2B), and platelets (FIG. 2C). The data are presented as the percentage of the baseline blood counts (mean=standard error of the mean; n=8/group). Statistical differences between the chemoprotectant groups compared with the rats given no chemoprotection is indicated by *, P<0.05; **, P<0.01; ***, P<0.001.

FIGS. 3A-3D show antitumor efficacy in the presence of chemoprotection. Nude rats with intracerebral tumors were untreated or treated with chemotherapy alone or in combination with chemoprotection consisting of N-acetylcysteine (1000 mg/kg, aortic infusion) 60 min before chemotherapy and/or sodium thiosulfate (8 g/m², intravenous administration) 4 and 8 h after tri-drug chemotherapy. Six days after treatment, rat brains were harvested for tumor volumetrics. FIG. 3A shows histology of untreated tumor. FIG. 3B shows histology of tumor after chemotherapy treatment. FIG. 3C shows histology of tumor after chemotherapy in combination with N-acetylcysteine and sodium thiosulfate. FIGS. 3A to 3C show 100-μm coronal sections with arrows indicating tumor; original magnification, 4×. FIG. 3D shows tumor volumes. All treatment groups were significantly different from the untreated controls; ***, P<0.0001. No significant differences were found comparing treatment groups with or without chemoprotection. Data are indicated as mean±standard deviation (n=8/group).

FIG. 4 shows effect of L-NAC and D-NAC on rat blood counts after chemotherapy.

### DETAILED DESCRIPTION OF THE INVENTION

The present invention provides methods for preventing or ameliorating chemotherapeutic agent-induced bone marrow toxicity (including thrombocytopenia). In certain embodiments, such methods do not substantially adversely affect the efficacy of chemotherapy. The prevention or amelioration of chemotherapeutic agent-induced bone marrow toxicity without substantially reducing efficacy of chemotherapy may be accomplished by spatial and/or temporal separation of the administration of chemoprotectant(s) from that of chemotherapeutic agent(s).

The methods of the present invention comprise administering to a patient in need thereof an effective amount of L-NAC. L-NAC has the following structure:

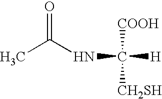

In certain embodiments, L-NAC administered is not present in a racemic mixture of L-NAC and N-acetyl-D-cysteine (D-NAC). In other words, in those embodiments, only L-NAC, not in mixture with equal amount D-NAC, is administered to a patient in need of chemoprotection to reduce bone marrow toxicity. In certain other embodiments, only L-NAC, not in mixture with any D-NAC, is administered to a patient in need of chemoprotection to reduce bone marrow toxicity. In certain other embodiments, L-NAC in a mixture containing less amount of D-NAC may be administered to a patient in need thereof. Such a mixture may contain, for example, about 95% L-NAC and about 5% D-NAC, about 90% L-NAC and about 10% D-NAC, about 80% L-NAC and about 20% D-NAC, about 70% L-NAC and about 30% D-NAC; about 60% L-NAC and about 40% D-NAC; about 55% L-NAC and about 45% D-NAC.

As used therein, “bone marrow toxicity” refers to the death or reduction of function of bone marrow cells due to chemotherapeutic agent(s).

“Chemotherapeutic agent” refers to a compound that is administered to a mammalian subject to destroy, or otherwise adversely affect, cancer cells. Chemotherapeutic agents include, but are not limited to, platinum derivatives (e.g., cisplatinum and carboplatinum), taxanes (e.g., paclitaxel), steroid derivatives, anti-metabolites (e.g., 5-fluorouracil, methotrexate and cytosine arabinoside), plant alkaloids (e.g., vindesine VP16, vincristine and vinblastine), antibiotics (e.g., adriamycin, mitomycin C, bleomycin, mithramycin, daunorubicin, mitoxantrone, and doxorubicin), etoposide, arsenic derivatives, intercalating agents, alkylating agents (e.g., melphalan, cyclophosphamide, chlorambucil, busulphan, thiotepa, isofamide, mustine, and the nitrosoureas), enzymes (e.g., asparaginase), biological response modifiers (e.g., immunoadjuvants and immunorestoratives), hydroxyurea, procarbazine, and combination thereof. In certain embodiments, chemotherapeutic agents are alkylating agents. In certain embodiments, alkylating agents include platinum-containing alkylating agents (e.g., cisplatin, carboplatin, and oxyplatin).

“Chemotherapeutic agent-induced bone marrow toxicity” (interchangeably used with “chemotherapy-induced toxicity”) includes thrombocytopenia caused or induced by the administration of a chemotherapeutic agent or a combination of chemotherapeutic agents.

“Preventing a chemotherapeutic agent-induced bone marrow toxicity” refers to preventing or diminishing the occurrence of chemotherapeutic agent-induced bone marrow toxicity. A subject in need of prevention of chemotherapeutic agent-induced bone marrow toxicity refers to a human, non-human primate or other mammal that will undergo, or is undergoing, chemotherapy an d is at high risk for chemotherapy-induced bone marrow toxicity.

A subject at high risk for chemotherapy-induced bone marrow toxicity is one that has at least one of the risk factors for chemotherapy-induced bone marrow toxicity. Such risk factors include previous bone-marrow depleting chemotherapy, performance status greater than 1, platelet count less than 150,000/μl at day 1 before the initiation of chemotherapy, lymphocyte count less or equate to 700/μl at day 1 before the initiation of chemotherapy, polymorphonuclear leukocyte count less than 1,500/μl at day 1 before the initiation of chemotherapy, and undergoing high risk chemotherapy (Blay et al., Blood 92: 405-10, 1998). High risk chemotherapy refers to regimens containing greater than 90 mg/m² doxorubicin, greater than 90 mg/m² epirubicin, greater than 100 mg/m² cisplatin, greater than 9 g/m² ifosfamide, greater than 1 g/m² cyclophosphamide, greater than 500 mg/m² etoposide, or greater than 1 g/m² cytarabine per course (Blay et al., Blood 92: 405-10, 1998; Blay et al., Proc. Am. Soc. Clin. Oncol. 16: 56a, 1997; Blay et al., J. Clin. Oncol. 14: 636, 1996). In certain embodiments, a subject in need of prevention of chemotherapeutic agent-induced bone marrow toxicity has one, two, three, four, five or more risk factors as described above.

“Ameliorating chemotherapeutic agent-induced bone marrow toxicity” refers to reducing the severity of chemotherapeutic agent-induced bone marrow toxicity. A subject in need of ameliorating chemotherapeutic agent-induced bone marrow toxicity refers to a human, non-human primate or other animal that is undergoing chemotherapy and suffers from chemotherapeutic agent-induced bone marrow toxicity.

The efficacy of a chemotherapeutic agent is not substantially adversely affected when the efficacy of the chemotherapeutic agent with administration of a chemoprotectant (e.g., L-NAC, L-NAC and STS) is at least about 75% of that without administration of the chemoprotectant. The efficacy of a chemotherapeutic agent may be measured by the reduction of tumor volume with administration of the chemotherapeutic agent compared to that without administration of the chemotherapeutic agent. In certain embodiments, the efficacy of chemotherapeutic agent with administration of a chemoprotectant is at least about 80%, 85%, 90%, 95% of that without administration of the chemoprotectant. In certain other embodiments, the efficacy of chemotherapeutic agent with administration of a chemoprotectant is about the same as, or even more than that without administration of the chemoprotectant.

“Thiol-based compound” refers to a compound containing a thio, thiol, aminothiol or thioester moiety. In certain embodiments, the thiol-based compounds have a free radical scavenging activity. The thiol-based compounds include, but are not restricted to, sodium thiosulfate, N-acetyl cysteine, glutathione ethyl ester, D-methionine, S-adenosyl-methionine, cysteine, N,N′-diacetyl-cysterine, cystathione, glutathione, glutathione ethyl ester, glutathione diethyl ester, S-(1,2-dicarboxyethyl) glutathione triester, cysteamine, cysteine isopropylester, and thiol amifostine (Ethyol or WR 2721). If a thiol-based compound contains one or more amino acid residues, the amino acid residues may be in L- or D-form. One or more thiol-based compounds may be used in conjunction with L-NAC, and/or other pharmaceutical agents and excipients.

“Thiol-based composition” refers to a composition comprising at least one thiol-based compound. Such compositions may also include, in addition to one or more thiol-based compounds, pharmaceutically acceptable carriers that facilitate administration of thiol-based compound(s)% to a mammalian subject.

L-NAC is administered “in conjunction with” another thiol-based compound (or composition) if at one time point, L-NAC and the other thiol-based compound (or composition) are co-present in at least one cell of the subject to which L-NAC and the other thiol-based compound are administered. In certain embodiments, L-NAC and another thiol-based compound (or composition) may be administered together to a subject. In certain other embodiments, L-NAC and another thiol-based compound (or composition) may be administered via the same administration route, but at different time points. In certain other embodiments, L-NAC and another thiol-based compound (or composition) may be administered via different administration routes at different time points.

In certain embodiments, the methods of the present invention comprise administering L-NAC and further administering another thiol-based compound or composition wherein the administration of L-NAC is not in conjunction with that of the other thiol-based compound or composition. However, the administration of the other thiol-based compound or composition provides additional chemoprotection against chemotherapeutic agent-induced bone marrow toxicity.

The term “effective amount” refers to an amount of a thiol-based compound (e.g., L-NAC) that is sufficient to prevent or reduce chemotherapeutic agent-induced bone marrow toxicity.

The present application provides thiol-based compositions and methods for using such compositions in preventing or ameliorating chemotherapy-induced bone marrow toxicity. Techniques for the formulation and administration of the compounds of the present application may be found in “Remington's Pharmaceutical Sciences” Mack Publishing Co., Easton, Pa., latest edition.

L-NAC and/or other thiol-based compounds are formulated to be compatible with their intended route of administration. Examples of route of administration include intravenous (i.v.), intra-arterial (i.a.), intra-peritoneal (i.p.), oral (p.o.), intradermal, subcutaneous, and transdermal administration. Solutions or suspensions used for intravenous, intra-arterial, intradermal, or subcutaneous application can include one or more of the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In addition, pH may be adjusted with acids or bases, such as hydrochloric acid or sodium hydroxide. The free radical scavengers are preferably administered in their un-oxidized form. The parenteral preparation can be enclosed in ampoules, disposable syringes or multiple dose vials made of glass or plastic.

L-NAC and other thiol-based compounds suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against contamination from microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In certain embodiments, a spatial two-compartment pharmacokinetic model is used to administrating L-NAC (and/or other thiol-based compounds) and chemotherapeutic agent(s). Any models known in the art that is suitable for spatially separating the chemotherapeutic agent(s) from chemoprotectants (e.g., L-NAC and other thiol-based compounds) may be used. Such separation allows for the reduction of chemotherapy-induced toxicity without affecting chemotherapy efficacy. Exemplary two-compartment pharmacokinetic models may be found in published PCT Application No. WO 01/80832. For instance, head and neck tumors are treatable through regionalization of chemotherapeutic agents to head and neck where the tumor tissue is located and through regionalization of chemoprotectants (e.g., L-NAC) to general tissues below the level of the heart where the majority of bone marrow tissue is located. An example of spatial compartmentalization is the administration of a chemoprotectant into the descending aorta or lower, preventing any significant chemoprotectant concentrations of the protectant from ever reaching head or neck where the tumor tissue is located. In certain other embodiments, spatial compartmentalization may be accomplished by delivering chemotherapeutic agent(s) via a major artery leading to the tumor tissue and delivering chemoprotectants (e.g., L-NAC) via a vein leading away from the tumor tissue.

It is advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent oh the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Various animal models and clinical assays for evaluating effectiveness of L-NAC and/or other thiol-based compounds in preventing or reducing bone marrow toxicity known in the art may be used in the present invention. They include, but are not limited to, those described in Das et al., Eur. J. Cancer 39: 2556-65, 2003; Beau et al., JPEN J. Parenter Enteral. Nutr. 21:343-6, 1997; Gibaud et al., Eur. J. Cancer 30A: 820-6, 1994; Blay et al., Blood 92: 405-10, 1998; Case et al., Stem Cells 18: 360-5, 2000; Issacs et al., J. Clin. Oncol. 15: 3368-77, 1997; Harker et al., Blood 89: 155-65, 1997. Additional assays are described in the example below.

The dosage of L-NAC useful in preventing or ameliorating bone marrow toxicity, when administered intra-arterially (or intravenously or via another route), may be about 200, 300, 400, 500, 600, 700, 800, 900, 1000, 1100, 1200, 1300, or 1400 mg/kg in humans, or a dosage in another subject comparable to that in humans. A dosage (“dosage X”) of a thiol-based compound in a subject other than a human is comparable to a dosage (“dosage Y”) of the thiol-based compound in humans if the serum concentration of the scavenger in the subject post administration of the compound at dosage X is equal to the serum concentration of the compound in humans post administration of the compound at dosage Y. In certain embodiments, L-NAC may be administered multiple times (e.g., 1, 2, 3, 4, 5, 6, 7, 8, 9, 10, br more times). In certain embodiments, L-NAC may be administered in conjunction with another thiol-based compound such as sodium thiosulfate.

L-NAC and/or other thiol-based compounds may be administered to a subject in need thereof prior to, concurrent with, or following the administration of chemotherapeutic agents. For instance, L-NAC may be administered to a subject at least about 15 minutes, 30 minutes, 45 minutes, 60 minutes, 1.5 hours, 2 hours, 3 hours, 4 hours, or any time between these values before the starting time of the administration of at least one chemotherapeutic agent. In certain embodiments, L-NAC and/or other thiol-based compounds may be administered concurrent with the administration of chemotherapeutic agent(s). In other words, in these embodiments, L-NAC and/or other thiol-based compounds are administrated at the same time when the administration of one or more chemotherapeutic agents start. In other embodiments, L-NAC and/or other thiol-based compounds may be administered following the starting time of administration of chemotherapeutic agent(s) (e.g., at least about 30 minutes, 1 hour, 2 hours, 3 hours, 4 hours, 5 hours, 6 hours, 7 hours, 8 hours, or any time between these values after the starting time of administration of chemotherapeutic agents). Alternatively, thiol-based compounds may be administered at least about 30 minutes, 1 hour, 2 hours, 3 hours, 4 hours, 5 hours, 6 hours, 7 hours, 8 hours, or any time between these values after the completion of administration of chemotherapeutic agents. Generally, L-NAC and/or other thiol-based compounds are administered for a sufficient period of time so that bone marrow toxicity is prevented or reduced. Such sufficient period of time may be identical to, or different from, the period during which chemotherapeutic agent(s) are administered. In certain embodiments, multiple doses of L-NAC and/or other thiol-based compounds are administered for each administration of a chemotherapeutic agent or a combination of multiple chemotherapeutic agents.

In certain embodiments, the methods of the present invention further comprise administering to a patient in need of chemoprotection against bone marrow toxicity an effective amount of STS. STS may be administered intravenously, intra-arterially, or via other routes. The dosage of STS may be at least about 1, 2, 3, 4, 5, 6, 7, 8, 9, 10, 11, 12, 13, 14, 15, 16, 17, 18, 19, or 20 g/m² in humans. In certain embodiments, multiple doses of STS may be administered. STS may be administered prior to, concurrent with, or subsequent to, the administration of chemotherapeutic agents. For instance, STS may be administered at least about 8, 7, 6, 5, 4, 3, 2, 1 hour, 30 minutes, or immdicately prior to, or subsequent to, the administration of chemotherapeutic agent(s)

In certain embodiments, an appropriate dosage of L-NAC is combined with a specific timing and/or a particular route to achieve the optimum effect in preventing or reducing bone marrow toxicity. For instance, L-NAC may be administered to a patient at about 1000, 1100, or 1200 mg/kg by aortic infusion about 30, 45, or 60 minutes prior to the administration of a chemotherapeutic agent or a combination of chemotherapeutic agents (e.g., carboplatin, melphalan and etoposide phosphate). In certain embodiments, STS may be further administered to the patient at about 15 to 20 g/m² about 4 hours to about 8 hours subsequent to the administration of the chemotherapeutic agent(s). In certain embodiments, multiple doses of STS may be administered, such as 20 g/m² about 4 hours after the administration of the chemotherapeutic agent(s) followed by 16 g/m² about 8 hours after the administration of the chemotherapeutic agent(s).

### EXAMPLES

### Example 1

This example evaluated whether an optimized bone marrow chemoprotection regimen impaired the efficacy of enhanced chemotherapy against rat brain tumors. Nude rats with intracerebral human lung carcinoma xenografts were treated with carboplatin, melphalan, and etoposide phosphate delivered intra-arterially with osmotic blood-brain barrier disruption (n=8/group). Thiol chemoprotection was N-acetyl-L-cysteine (1000 mg/kg) 60 min before chemotherapy and/or sodium thiosulfate (8 g/m²) 4 and 8 h after chemotherapy, when the blood-brain barrier is reestablished. Blood counts were obtained before treatment on day 3 and at sacrifice on day 9. N-acetylcysteine serum clearance half-life was 9 to 11 min. Pretreatment with N-acetylcysteine combined with delayed administration of sodium thiosulfate protected against toxicity toward total white cells, granulocytes, and platelets (P=0.0016). Enhanced chemotherapy reduced intracerebral tumor volume to 4.3±1.0 mm³ compared with 29.1±4.1 mm³ in untreated animals (P<0.0001). Tumor volume was 3.7±0.6 mm³ in rats that received N-acetylcysteine before and sodium thiosulfate after chemotherapy. The data indicate the efficacy of enhanced chemotherapy for rat brain tumors was not affected by thiol chemoprotection that provided excellent protection for hematological toxicity. Negative interactions of thiols with antitumor efficacy were avoided by temporal and spatial separation of chemoprotectants and chemotherapy.

Materials and Methods

Animal studies were performed in accordance with guidelines established by the Oregon Health Sciences University Committee on Animal Care and Use.

Osmotic Blood-Brain Barrier Disruption. Anesthesia was induced with 5% isoflurane and maintained with propofol (650 μg/kg/min). Mannitol (25%, 37° C.) was infused cephalad into the left internal carotid artery via a left external carotid catheter (Remsen et al., Anesthesia Analgesia 88:559-67, 1999).

Aortic Infusion Technique. The left internal carotid artery was temporarily occluded, and agents were administered retrograde to the descending aorta through a left external carotid catheter (Neuwelt et al., Cancer Res 61:7868-74, 2001).

N-Acetylcysteine Toxicity. N-Acetyl-L-cysteine (N-acetylcysteine, Mucomyst; Roxane Laboratories, Inc., Columbus, Ohio) was given by aortic infusion 30 min (n=7) or 60 min (n=10) before blood brain barrier disruption in normal Long Evans rats. Doses ranged from 400 to 1500 mg/kg in 3 ml infused at 0.6 ml/min. Rats were sacrificed 6 days after treatment or at signs of acute neurotoxicity (head tilt, circling, moribund).

N-Acetylcysteine Clearance. Rats were treated as follows: group A, 1200 mg/kg aortic infusion (n=3); group B, 1000 mg/kg aortic infusion (n=3); group C, 400 mg/kg administered intravenously (n=4); and group D, 140 mg/kg by aortic infusion (n=2). Blood samples (0.5 ml) were collected 5, 15, 30, 60, and 90 min after thiol administration, and serum was evaluated for N-acetylcysteine concentration. Serum N-acetylcysteine concentrations were measured using the Bioxytech GSH-400 colorimetric kit (Oxis Research, Portland, Oreg.). The colorimetric assay was validated by high-pressure liquid chromatography (HPLC) analysis of serum thiols for n=2 rats from groups B and D. Deproteinated serum samples were diluted in 160 mM KH₂PO₄, pH 3. Thiols were measured by electrochemical detection using a Waters radial compression module with 10-μm C18 column (Waters, Milford, Mass.), an ESA 5010 analytical cell, and an ESA 5100A coulochem detector (ESA Inc., Chelmsford, Mass.). Area under the curve was compared with known concentrations prepared in control sera.

Pilot Studies of Chemoprotection. Pilot study 1 evaluated the timing for platelet protection with sodium thiosulfate. Normal Long Evans rats (n=24, six rats per group) received intravenous 8 g/m² sodium thiosulfate (Sigma-Aldrich, St. Louis, Mo.) 2, 4, or 8 h after administration of 800 carboplatin (Paraplatin; Bristol-Myers Squibb Co., Stamford, Conn.). Pilot study 2 evaluated various timing schemes for chemoprotection. Normal rats (n=54, six rats per group) were treated with a tri-drug chemotherapy regimen consisting of carboplatin (200 mg/m²), melphalan (10 mg/m² Alkeran; GlaxoSmithKline, Uxbridge, Middlesex, UK), and etoposide phosphate (100 mg/m² Etopophos; Bristol-Meyers Squibb Co.), administered in the right carotid artery. Sodium thiosulfate (8 g/m²) was administered intravenously 4 and/or 8 h after chemotherapy, either alone or in combination with N-acetylcysteine (1200 mg/kg, aortic infusion) 30 min before chemotherapy. For both pilot studies 1 and 2, blood counts were determined at 6 days after chemotherapy, in comparison with untreated controls. For blood count analysis, 0.5 ml of whole blood collected in EDTA microtubes was analyzed in duplicate on a Hemavet 850 (CDC Technologies Inc., Oxford, Conn.).

Tumor Studies. Female athymic nude rats (rnu/rnu, 200-220 g) were anesthetized with intraperitoneal ketamine (60 mg/kg) and diazepam (97.5 mg/kg). LX-1 human small cell lung carcinoma cells (1×10⁶ cells in 12 μl, >90% viability) were inoculated stereotactically in the left caudate putamen (vertical bregma—6.5 mm, 3.1 mm lateral).

In pilot study 3, tumor-bearing rats (n=24) were treated with the tri-drug chemotherapy regimen 3 days after tumor implantation. Rats were sacrificed 12 days after treatment or earlier, if toxicity warranted.

For the major study, 40 rats (eight rats per group) were treated 3 days after tumor implantation. Rats received either no treatment, tri-drug chemotherapy, or chemotherapy in combination with N-acetylcysteine (1000 mg/kg, aortic infusion) 30 min before chemotherapy and/or sodium thiosulfate (8 g/m², intravenous) 4 and 8 h after chemotherapy (n=8/group). The tri-drug intra-arterial chemotherapy regimen consisted of etoposide phosphate (100 mg/m²) given immediately before blood-brain barrier disruption and carboplatin (200 mg/m²) and melphalan (10 mg/m²) immediately after blood brain barrier disruption. Blood counts were obtained as described above at baseline (prechemotherapy) and at 6 days after treatment. Rats were then sacrificed by barbiturate overdose, and the brains fixed by immersion in 10% formalin for vibratome sectioning (100-μm coronal sections). Every sixth brain section was stained with hematoxylin then imaged at high resolution on an Epson 1640XL flatbed scanner using Adobe Photoshop software. Tumor volume was assessed using NIH Image software.

Statistical Analysis. Least-squares means were estimated for blood counts and changes from each animal's baseline values. A mixed model repeated measures analysis of variance was performed using group as one factor and time (pre versus post) as the second (repeated) factor (SAS version 8.01; SAS Institute Inc., Cay, N.C.). A Wilcoxon rank sums analysis was performed to evaluate the change from baseline values in all groups as well as each chemoprotection group in comparison with the untreated controls with a Bonferroni adjustment. P values were determined using the Kruskal-Wallis test. An analysis of variance test was also performed on the change from baseline values, with similar results, but only the P values from the Wilcoxon analysis are shown because the high variability in the blood data reduces the assumption of normalcy.

For the analysis of tumor volume, a one-way analysis of variance model was fit to the data. The assumptions for this analysis include an approximate normal distribution and equal variances across groups. To meet these assumptions, the square-root transformation was applied to these data. The least-square means were estimated, and differences among these means were tested with Tukey adjustment for multiple testing. Nonparametric analyses (a Kruskal-Wallis test with pairwise comparison of means with a Bonferroni adjustment) were also performed with similar results.

Results

N-Acetylcysteine Toxicity and Clearance. In a previous study of bone marrow chemoprotection with thiols, N-acetylcysteine was administered at a dose of 1200 mg/kg 30 min before chemotherapy, using an aortic infusion technique (Neuwelt et al, Cancer Res 61:7868-74, 2001). This regimen was neurotoxic in combination with blood-brain barrier disruption. Therefore, both a reduction in the N-acetylcysteine dose and an increase in the time before barrier opening (n=17) were evaluated. The maximum tolerated dose was 500 mg/kg 30 min before blood-brain barrier disruption, and 1000 mg/kg 60 min before blood-brain barrier disruption.

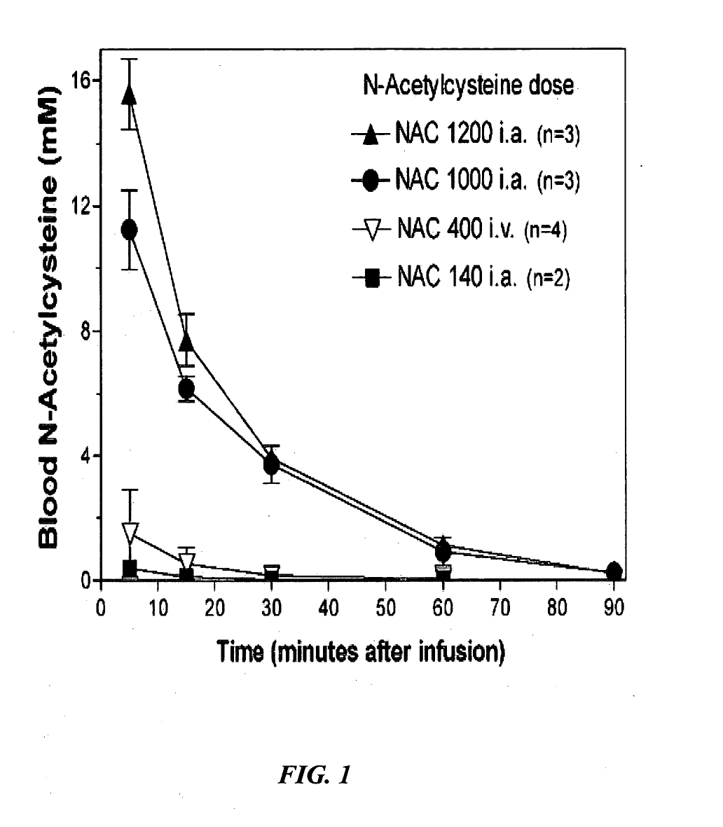

The clearance of N-acetylcysteine from blood was evaluated in normal rats given high-dose or low-dose N-acetylcysteine via intravenous or aortic infusion routes of administration (FIG. 1). In all groups, N-acetylcysteine was cleared with a half-life of approximately 9 to 11 min, similar to the previously reported 15 min half-life for sodium thiosulfate (Neuwelt et al., J Pharmacol Exp Ther 286:77-84, 1998). In rats-given 1000 mg/kg N-acetylcysteine intra-arterially, the maximum blood concentration 5 min after infusion was 11.2±1.3 mM (FIG. 1), whereas blood concentration at the time of chemotherapy delivery (60 min after infusion) was 0.2±0.1 mM. The colorimetric assay for F1 N-acetylcysteine was validated by an HPLC assay of N-acetylcysteine and other thiols. Table 1 indicates that there was close correlation of these two assays, at both low and high serum N-acetylcysteine concentrations.

TABLE 1
Correlation of N-acetylcysteine colorimetric and HPLC assays

| N-Acetylcysteine Dose and | Colorimetric | HPLC |
|---|---|---|
| Route of Administration | Assay | Assay |
| 1000 mg/kg, aortic infusion | 7.1 | 5.6 |
| | 12.6 | 10.8 |
| 140 mg/kg, intravenous | 0.08 | 0.07 |
| | 0.29 | 0.18 |

Individual rat serum samples obtained 5 min after administration of N-acetylcysteine were analyzed by both assays; data are indicated in millimeter concentration.

Effect of Thiols on Chemotherapy-Induced Bone Marrow Toxicity. Pilot studies were performed to evaluate thiol timing and combination regimens to maximize chemoprotection. Previously, it was showed that sodium thiosulfate had minimal bone marrow chemoprotective activity either alone or in combination with N-acetylcysteine, when it was administered immediately after chemotherapy (Neuwelt et al., Cancer Res 61:7868-74, 2001). Pilot study 1 assessed the effect of sodium thiosulfate given 2, 4, or 8 h after high-dose carboplatin. The data suggested that delaying sodium thiosulfate administration improved platelet chemoprotection. In a second pilot study, delayed sodium thiosulfate was evaluated for bone marrow chemoprotection with or without a 30-min pretreatment with high-dose N-acetylcysteine. Tri-drug chemotherapy alone reduced platelet counts from 837±298 to 152±78 thousand/μl (mean=standard deviation, n=6/group). In rats treated with tri-drug chemotherapy in combination with N-acetylcysteine (1200 mg/kg by aortic infusion 30 min before chemotherapy) and sodium thiosulfate (8 g/m² given intravenously 4 and 8 h after chemotherapy), platelet counts were 475±289 thousand/μl. Due to the high variability of the platelet counts, limited animal numbers per group and the Bonferroni adjustment for testing nine pilot groups, the result was not significant. These pilot studies allowed the narrowing down of the groups in the current study and the evaluation of whether thiol pretreatment, delayed treatment, or both, would impact antitumor efficacy, when leakage into tumor was maximized with osmotic blood-brain barrier opening.

The tri-drug chemotherapy regimen (carboplatin, melphalan, and etoposide phosphate) caused significant mortality. In a third pilot study in tumor-bearing nude rats treated with tri-drug chemotherapy without chemoprotectants (n=24), deaths occurred on day 6 (n=7) and day 7 (n=7) after treatment. Mortality may be due to a number of contributing toxicities, including mucositis and resultant dehydration and weight loss, liver and kidney toxicity, and bone marrow toxicity, as well as complications related to the intracerebral tumor. Survival of untreated tumor-bearing rats averages 15 days (Remsen et al., Neurosurgery 46:704-709, 2000). These data demonstrated that survival was an inappropriate measure of antitumor efficacy of the chemotherapy regimen because in the absence of chemoprotection the rats died from the treatment itself. We have previously shown that the blood count nadir occurred at approximately 6 days after chemotherapy treatment, and blood counts recovered to above baseline by 9 to 12 days. Thus, in the tumor study, the animals were sacrificed for blood count and tumor volume measurements at 6 days after chemotherapy (9 days after tumor implantation). At this time point, total white cells were reduced to 1.24±0.70 thousand/μl from a baseline of 2.95±0.95 thousand/μl (n=8; P=0.0018), granulocytes were reduced to 60.86±0.53 from 2.39±±0.86 thousand/μl (n=8; P=0.0009), and platelets were reduced to 221±107 from 716±61 thousand/μl (n=8; P<0.0001).

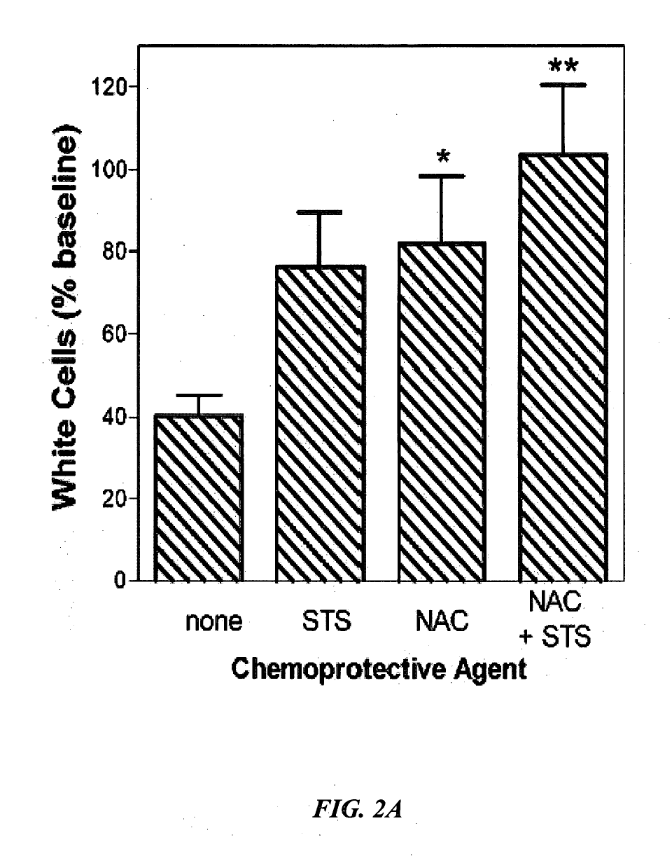

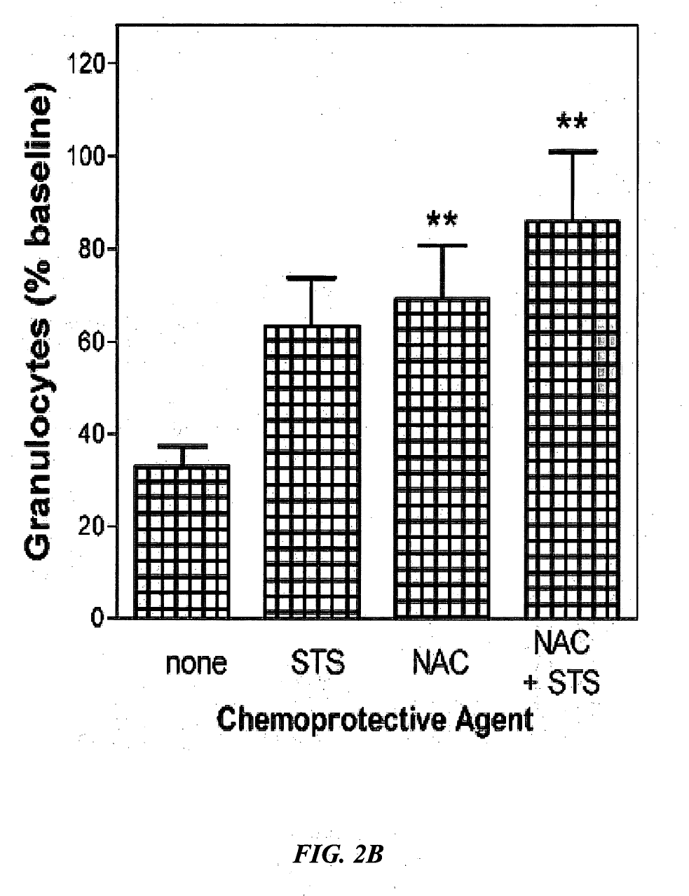

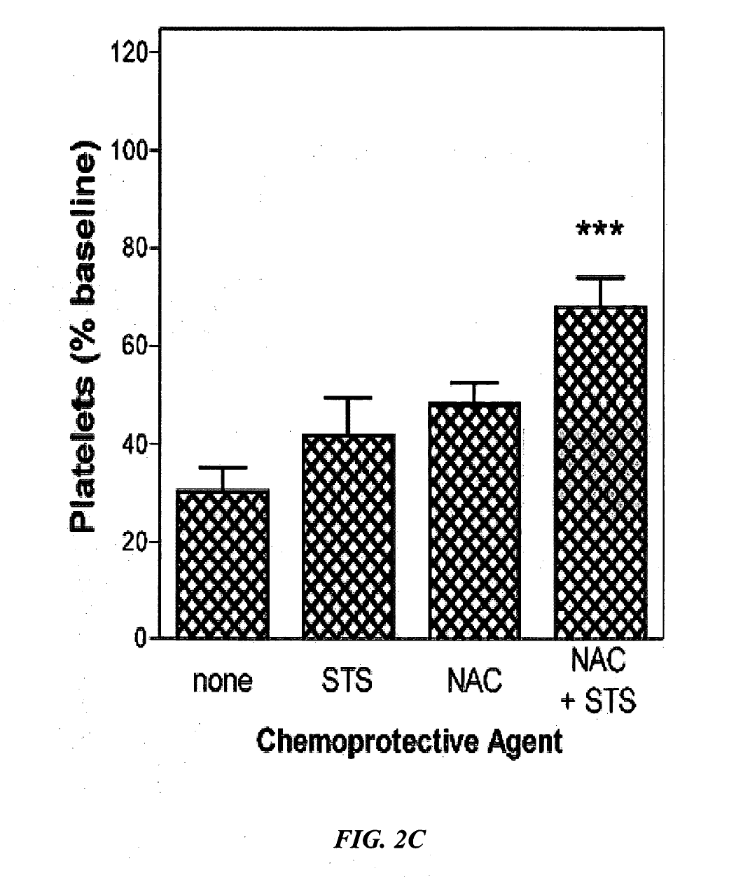

Thiol treatment provided bone marrow chemoprotection (FIG. 2). Delayed administration of high-dose sodium thiosulfate (8 g/m², 4 and 8 h after chemotherapy) had minimal protective effect against chemotherapy-induced bone marrow suppression (P>0.05). Pretreatment with N-acetylcysteine (1000 mg/kg by aortic infusion, 60 min before chemotherapy) was significantly protective for white cells (FIG. 2A; P=0.0117) and granulocytes (FIG. 2B; P=0.0087). Platelet chemoprotection was not significant with N-acetylcysteine alone. The best blood chemoprotection, particularly for platelets, was found combining both pretreatment with N-acetylcysteine and delayed treatment with sodium thiosulfate. With this dual chemoprotection approach, tri-drug chemotherapy-induced blood count nadirs were 104±48% of baseline for total white cells (2.58±0.93 thousand/μl; P=0.0029 compared with no chemoprotection), 86±43% of baseline for granulocytes (1.68±0.62 thousand/μl; P=0.0050), and 68±1% of baseline for platelets (478±139 thousand/μl; P=0.0002).

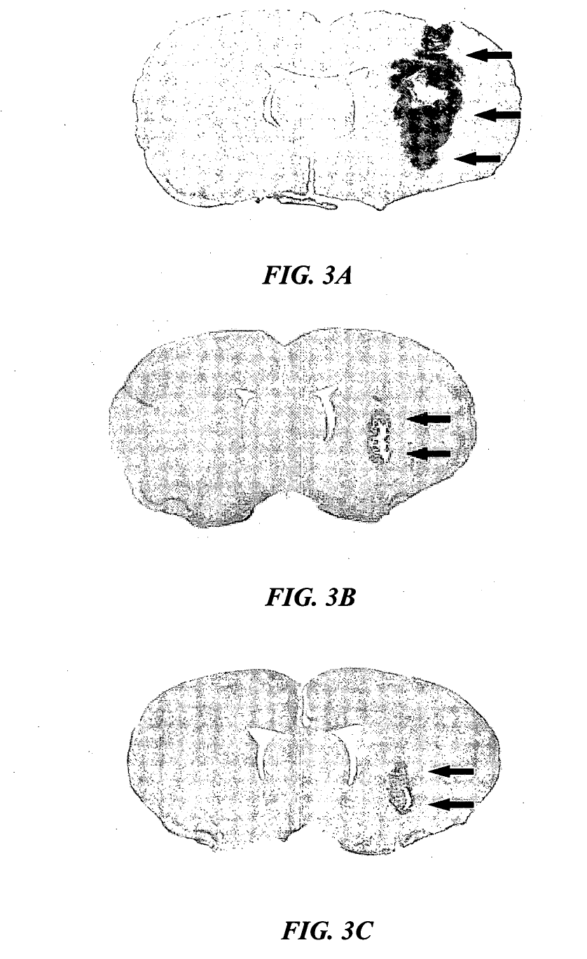

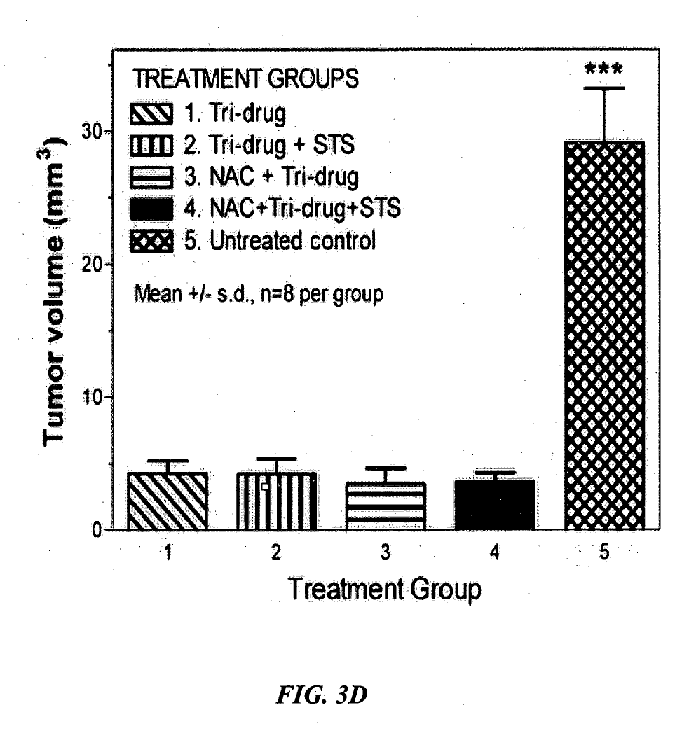

Effect of Thiols on Chemotherapy Efficacy. LX-1 small cell lung carcinoma intracerebral xenografts grew rapidly in nude rats, attaining a volume of 29.1±4.1 mm³ in untreated animals (range 24.2-34.8 mm³; FIG. 3A). The tridrug chemotherapy regimen was highly effective administered intra-arterially with blood-brain barrier disruption 3 days after tumor implantation (FIG. 3B), and this was not altered by chemoprotection (FIG. 3, C and D). Tri-drug chemotherapy treatment reduced intracerebral tumor volume to 4.3±1.0 mm³ (range 3.1-5.9 mm³; n=8; P<0.0001). The differences between each randomized active treatment group (+chemotherapy) and the untreated control were all significant (P<0.0001). By contrast, there was no difference in tumor volume between any of the groups that received chemotherapy, whether or not they also received chemoprotection. Even in the most aggressive chemoprotection group, with N-acetylcysteine 60-min pretreatment and sodium thiosulfate 4 and 8 h after treatment, tumor volume was 3.7±0.6 mm³ (range 2.7-4.7 mm³; n=8; FIG. 3D).

### Example 2

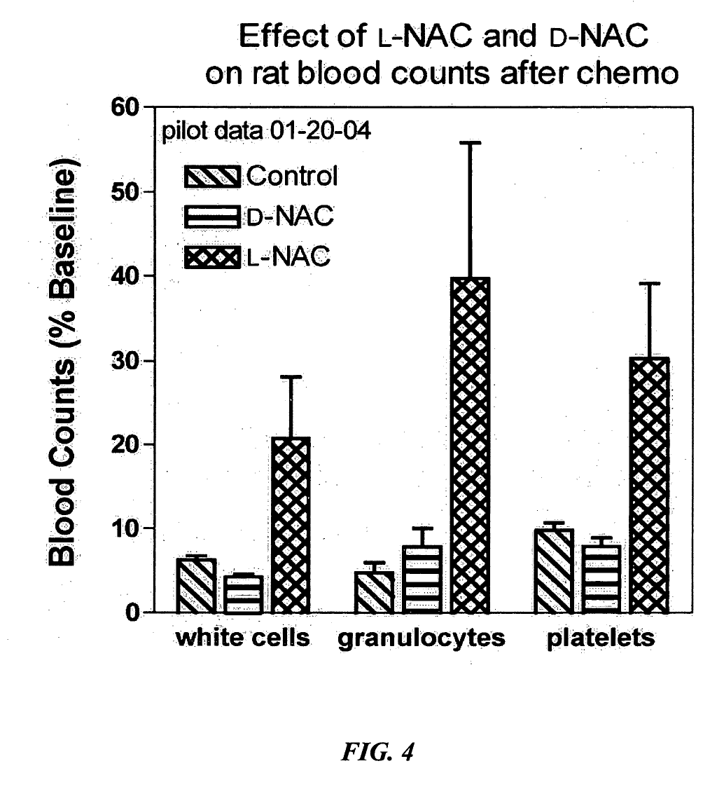

This example shows that L-NAC, not D-NAC, has a bone marrow chemoprotective activity. In the experiment shown in FIG. 4, rats were pretreated With buthionine sulfoximine to enhance the bone marrow toxicity of chemotherapy, and then treated with the tri-drug chemotherapy regimen with or without L-NAC or D-NAC. Pretreatment with the D-isomer of N-acetylcysteine is not bone marrow protective, while pretreatment with L-NAC significantly reduced chemotherapy toxicity to total white cells, granulocytes, and platelets.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for preventing or ameliorating chemotherapeutic agent-induced bone marrow toxicity, comprising administering to a patient in need thereof an effective amount of N-acetyl-L-cysteine (L-NAC) having the following structure: 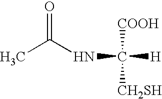
prior to, concurrent with, or subsequent to, the administration of a chemotherapeutic agent or chemotherapeutic agents, wherein L-NAC is not present in a racemic mixture of n-acetylcysteine (NAC).

2. The method according to claim 1 wherein L-NAC is administered prior to the administration of the chemotherapeutic agent or chemotherapeutic agents

3. The method according to claim 1 wherein L-NAC is administered intra-arterially.

4. The method according to claim 1 wherein L-NAC is administered prior to the administration of at least one of the chemotherapeutic agents.

5. The method according to claim 1 wherein L-NAC is administered about 30 minutes prior to the administration of the chemotherapeutic agent or at least one of the chemotherapeutic agents.

6. The method according to claim 1 wherein L-NAC is administered about 60 minutes prior to the administration of the chemotherapeutic agent or at least one of the chemotherapeutic agents.

7. The method according to claim 1 wherein L-NAC is administered intravenously.

8. The method according to claim 1 wherein the chemotherapeutic agent is an alkylating agent.

9. The method according to claim 1 wherein the alkylating agent is a platinum-containing alkylating agent.

10. The method according to claim 8 wherein the platinum-containing alkylating agent is selected from the group consisting of cisplatin, carboplatin, and oxyplatin.

11. The method according to claim 1 wherein the chemotherapeutic agents comprise melphalan, carboplatin and etoposide phosphate.

12. The method according to claim 1 wherein the patient in need thereof has a tumor in the head or neck.

13. The method according to claim 1 wherein the patient in need thereof is a human.

14. The method according to claim 13, wherein L-NAC is administered about 1,000 mg/kg to about 1,400 mg/kg.

15. The method according to claim 1 further comprising administering an effective amount of sodium thiosulfate (STS).

16. The method according to claim 15 wherein STS is administered intravenously.

17. The method according to claim 15 wherein STS is administered intra-arterially.

18. The method according to claim 15 wherein STS is administered subsequent to the administration of at least one of the chemotherapeutic agents.

19. The method according to claim 15 wherein STS is administered about 4 hours subsequent to the administration of at least one of the chemotherapeutic agents.

20. The method according to claim 15 wherein STS is administered about 8 hours subsequent to the administration of at least one of the chemotherapeutic agents.

21. The method according to claim 15 wherein STS is administered at a dosage of about 15 g/m² to about 20 g/m².

22. The method according to claim 1 wherein the efficacy of the chemotherapeutic agent(s) is not adversely affected.

23. A composition comprising L-NAC and a pharmaceutical acceptable carrier, adapted for preventing or ameliorating chemotherapeutic agent-induced bone marrow toxicity, wherein the composition does not contain equal amount of D-NAC.

24. The composition of claim 23, wherein L-NAC is present in a mixture with D-NAC and the mixture contains about 55% to about 95% of L-NAC and about 45% to about 5% of D-LAC.
